(19) United States
(12) Patent Application Publication
(10) Pub. No.: US 2008/0008699 A1
(43) Pub. Date: Jan. 10, 2008

(54) Method for proteomic analysis utilizing immune recognition and cumulative subtraction

(76) Inventors: Jianxun Li, Highland Park, IL (US); Tianquan Jin, Chicago, IL (US); Kangcheng Ruan, Shanghai (CN)

Correspondence Address: Olson & Cepuritis, LTD., 20 NORTH WACKER DRIVE, 36TH FLOOR, CHICAGO, IL, 60606, US

(21) Appl. No.: 11/586,189

(22) Filed: Oct. 25, 2006

Related U.S. Application Data
(60) Provisional application No. 60/731,099, filed on Oct. 28, 2005.

(51) Int. Cl. A61K 39/395; A61K 39/00; A61P 43/00; C07K 14/00; C07K 16/18; G01N 33/48

(52) U.S. Cl. 424/130.1; 424/184.1; 436/63; 530/350; 530/387.1

(57) ABSTRACT

A method of identifying proteins in a proteome comprises the steps of: (a) immunizing a host organism with a sample containing an initial mixture of proteins from a tissue or fluid to be evaluated to elicit an antibody response to one or more proteins in the initial mixture; (b) isolating an antiserum from the host organism after immunization; (c) contacting the antiserum with an array of known proteins to form one or more antibody-protein conjugates, thereby identifying the one or more proteins in the initial mixture that elicited an antibody response in the host organism; (d) optionally removing the proteins that elicited antibody formation in step (a) from the initial mixture to form a mixture of non-responding proteins; and (e) optionally repeating the previous steps (a) through (d) one or more times, each repetition utilizing the mixture of non-responding proteins from the immediate prior repetition in place of the initial mixture of proteins to identify one or more additional proteins present in the tissue or fluid to be evaluated. A corresponding method of preparing antisera for identifying proteins in a proteome is also disclosed.

### CROSS-REFERENCE TO RELATED APPLICATION

This application claims the benefit of U.S. Provisional Application for Patent Ser. No. 60/731,099, filed on Oct. 28, 2005, which is incorporated herein by reference.

### STATEMENT OF GOVERNMENT INTEREST

This invention was made with governmental support from the United States Government, National Institutes of Health, Grant CAGM54715; the United States Government has certain rights in the invention.

### FIELD OF THE INVENTION

This invention relates to proteomics. More particularly, this invention relates to methods for identifying proteins within a given proteome utilizing immune recognition and cumulative subtraction techniques.

### BACKGROUND

Proteomics can be summarized as the study of the composition, quantification, and functions of any given pool of proteins in any specific tissue or body fluid in health and in disease¹. This knowledge has applications not only in basic research such as gene functions, cell phylogeny, tissue specificity, and systematic biology, but also in clinical research applications including diagnosing diseases, identifying therapeutic markers and targets, and profiling of response to toxins and pharmaceuticals².

Although cDNA array is widely used to investigate the mRNA level, the transcription level of mRNA is not always a true reflection of the expression level of corresponding proteins^{3,4}. Especially in the case of body fluids such as saliva, mRNA level does not apply. The proteins in these proteomes have to be studied directly.

A number of techniques have been used. Fast developing mass spectrometry (MS)^{5,6} in combination with traditional biochemical separation techniques such as two-dimensional gel electrophoresis (2DE) or liquid chromatography (LC) has been successful in solving the proteome of many simple systems, including midbody⁷ and nuclear pore complex (NPC)⁸, and is being applied to more complex systems, including saliva⁹⁻¹¹, serum^{12,13} and others¹⁴⁻¹⁶. MS combined with affinity enrichment methods has also proven successful in screening several types of covalent modifications of proteins, including phosphorylation, glycosylation, farnesylation and ubiquitination (reviewed in Ref. 17).

However, the extraordinary capability of MS identification is heavily restricted by the capability of protein separation methods. Current separation methods for proteomics include mainly 2DE, LC and affinity enrichment. 2DE, the most widely used separation method for proteomics, has its intrinsic limitations. First, the separation capability of 2DE is about 1000-2000 proteins, much lower than the 12,000 proteins estimated in any given cell type (reviewed in Ref. 17). Second, the signals of the low abundant (<0.01%) proteins are overridden by the abundant proteins¹⁸, but large numbers of proteins in cells or body fluids are below this abundance. Third, membrane proteins are underrepresented in 2DE because of its solubility^{18,19,20}. Fourth, the proteins with extreme isoelectropoint and molecular weight cannot be separated by 2DE (reviewed in Ref. 21). LC separation is more repeatable and automatic. However, its separation capability still cannot meet the requirement for most proteomes. The difficulty imposed by low abundant proteins cannot be overcome by LC either²². Affinity depletion is used to enrich the low abundant proteins, for example, albumin was immuno-depleted from serum before 2DE or LC separation^{23,24}, but the application is restricted by the limited number of known proteins in a proteome and the availability of antibodies. After the depletion, there are still some proteins below the sensitivity of 2DE and LC assay.

The most restrictive limitation of these biochemical separations, however, is that they do not provide a progressive procedure for proteomics. Either the same person who runs a 2DE everyday, or different people in different laboratories who run 2DE in any day, will see a similar set of proteins within limitations, with little hope of identifying new proteins. Therefore, the data of separation-based proteomics are noncumulative, and an entire proteomics project is difficult to perform in parallel by sharing; people cannot build on each other's progress. The analyzed proteins cannot be eliminated so that those unknown ones can be enriched. On the other hand, data from different laboratories cannot be linked logistically and accumulated systematically. When a different biochemical separation technique is used, there is no logical link to the data obtained previously with the 2DE method. In the end, we will not know where the ending point is and how many alternative separation systems should be applied to ensure that all the proteins are identified. Therefore, it is necessary to develop new methods with which proteomes can be studied progressively.

Revisiting the success of human genomic project, one of the important factors is that tasks were divided along chromosomes, and data from every laboratory can be deposited into a database so that experiments will not be redundant. This is exactly what is lacking in current proteomics methods. There is an ongoing need and desire for a proteomics method that provides for increased rapidity in the identification of proteins in a proteome. There is also an ongoing need for proteomic methods that provide for division of protein identification tasks into multiple and non-overlapping portions to be distributed among a plurality of individuals or laboratories over potentially wide ranging distances. The methods of the present invention fulfill these needs.

### SUMMARY OF THE INVENTION

While composition, quantification and functions are three major aspects of current proteomics methods, identifying the composition of any given proteome alone is still a challenge, mostly because of the limitation of biochemical separation techniques. Based on the immune system's capability of identifying vast numbers of foreign proteins and each antigen eliciting a corresponding antibody, combined with high throughput analysis of protein microarray, the present invention provides an antibody-based cumulative subtraction proteomics (ABCSP) method.

In this method, protein signals in a sample of tissue or fluid from any given proteome are converted to antibody signals by immunization of a host organism with a sample containing the entire protein mixture present in the tissue or fluid to be evaluated or any portion of the protein mixture. The proteins in the sample that have elicited antibody formation in a first cycle of immunization in a first host organism are identified, e.g., by immunoblotting a protein array (e.g., a chip) containing addressed known proteins with the antiserum generated by immunization of the host organism. The identified antibody-eliciting proteins of the first cycle of immunization preferably are then immunodepleted from the total protein mixture in the sample, e.g., by antiserum affinity chromatography (i.e. the antibody-eliciting proteins are “subtracted” from the mixture). The non-antibody-eliciting proteins remaining in the sample after the first cycle of immunization are thereby enriched and preferably are then subjected to one or more additional cycle(s) of immunization in one or more host organism(s). Eventually, after several cycles of immunization, identification and subtraction, most proteins of the proteome are identified. This method provides for the identification of any proteome to proceed cumulatively in parallel at multiple locations, thus providing a platform for a world-wide collaboration to determine the composition of any proteome.

In one embodiment, the method of the present invention provides for identifying the composition of the human saliva proteome. In one example of the method of this embodiment, 14 proteins in a mixture of 18 were identified in two cycles of ABCSP. When this system was applied to a whole saliva proteome with an antigen chip protein array containing 504 different known proteins, 36 previously unreported saliva proteins were identified.

In principle, the immune system has the ability to recognize a vast number of foreign proteins and produce antibodies against them. When proteins in a sample of a given proteome are used to immunize animals of a given species, a portion of the proteins present in the sample will elicit antibodies in the animals based on a combination of antigenicity and concentration of the individual proteins in the sample. The antibodies elicited against proteins in the sample are identified, e.g., by immunoblotting a protein array (e.g., a protein chip) containing a plurality (preferably a relatively large number, e.g., hundreds) of known proteins, which consequently identifies the existence of proteins in the proteome that correspond to known proteins in the array.

Of course, there must be some proteins that do not elicit antibodies in the first round of immunization. For clarity, we have termed the proteins that have elicited antibodies in a round of immunization as “antibody-eliciting proteins”. Those proteins that have not induced detectable antibody are thus referred to as “nonantibody-eliciting proteins.” Those nonantibody-eliciting proteins in the first round of immunization could be enriched simply by immunodepletion of those antibody-eliciting proteins from the total protein mixture with the antiserum from the first round of immunization. Those enriched proteins will be subjected to a new round of immunization and more antibody species will be elicited. Further cycles of immunization of a naive host, and immunoblotting the same or different protein arrays can result in identification of all proteins in a given proteome, or a desired percentage thereof. Proteins remaining in a sample after a number of cycles that do not correspond to any known proteins in a protein array can be identified by other methods known in the art. In this way proteins of an uncharacterized proteome, or in a partially characterized proteome, can be identified rapidly and efficiently.

An outline of the method is illustrated in FIG. 1.

Most proteins in a proteome can be identified upon accumulation of the data after a number of cycles of immunization, immunoblotting of protein arrays, immunodepletion and re-immunization. Thus, a set of antisera can be developed against any proteome of interest and used to test any protein to determine whether it exists in the proteome. The expression work and detection work can be carried out by different individuals or groups in different locations so long as a standardized set of serum for a given proteome is distributed among the different individuals, even at very disperse geographic locations. Each individual or group can screen a set of proteins, and eventually, results from each individual or group can easily be accumulated, allowing the assembly of the entire proteome.

This new system, which is termed antibody-based cumulative subtraction proteomics (ABCSP), was applied to saliva proteomics. Saliva has the potential to provide biomarkers for many diseases such as Sjogren's syndrome²⁵, rheumatoid arthritis²⁶, alcoholic cirrhosis²⁷, cystic fibrosis²⁸, diabetes mellitus²⁹, diseases of the adrenal cortex³⁰, cardiovascular diseases³¹ and dental caries³². Together with the advantage of simple and noninvasive collection, saliva diagnosis appears to hold promise for the future³³.

### BRIEF DESCRIPTION OF THE DRAWINGS

FIG. 1. Outline of the procedures for ABCSP.

FIG. 2. The microarray of the 18 known proteins. Each protein was serially diluted by fourfold and printed on a glass slide at the same column. A, Immunoblotting with the first round of antiserum against the mixture of 18 known proteins. The left panel is a scanned protein chip picture, the pseudo colors of blue, green, yellow, red and white represent the signal intensity from low to high; the right panel is a bar graph of the signal intensity of the highest concentration spots (first row), the same experiment was repeated 4 times. The signal above 8000 was considered positive. B is the same experiment as A, except that in the second round, instead of the first round, antiserum were used.

FIG. 3. Characterization of the antiserum against whole saliva proteins with 2DE gel Western blot. 2DE was run at the pH range 3-10 for the first dimension and 14% of SDS-PAGE for the second dimension. The yellow line marked the area where the spots were not separated well on Western blot. The red “O” marked the separated proteins on silver staining and their corresponding spots on Western blot; The green “O” marked the proteins only detected by Western blot; The “Δ” marked the proteins detected by silver staining but not detected by Western blot. (A) Silver staining of the 2DE gel; (B) The parallel 2DE gel was transfered to PVDF membrane and Western blotted with the antiserum against whole saliva proteins.

FIG. 4. Western blot of 22 known saliva GST fusion proteins with the antiserum against whole saliva proteins. The negative proteins were marked with *. 1, AMY2B; 2*, B2M; 3, S100A8; 4, S100A9; 5, ENO1; 6, ACATE2; 7,FABP1; 8, FABP3; 9, FABP4; 10, FABP5; 11, FABP6; 12, FABP7; 13,GSTA1; 14, GSTA4; 15*, KLK1; 16*, KLK2; 17*, KLK8; 18, LIPA; 19, LYZ; 20, PIP; 21,PRL; 22,Hbb; B, BL21; S, Saliva.

FIG. 5. The protein chip containing 504 randomly selected GST proteins and 22 known saliva GST fusion proteins immunoblotted with the anti-saliva serum. Total saliva proteins at (V,1) and bacteria lysate at (V,13) as positive and negative controls, respectively. A, immunoblotted with the first round of antisaliva IgG pool; B, immunoblotted with the second round of antisaliva IgG pool, the new positive proteins were marked with a red circle.

### DETAILED DESCRIPTION OF PREFERRED EMBODIMENTS

A method of identifying proteins in a proteome comprises the steps of: (a) immunizing a host organism with a sample containing an initial mixture of proteins from a tissue or fluid to be evaluated to elicit an antibody response to one or more proteins in the initial mixture; (b) obtaining an antiserum from the host organism after immunization; (c) contacting the antiserum with an array of known proteins to form one or more antibody-protein conjugates, thereby identifying the one or more proteins in the initial mixture that elicited an antibody response in the host organism; (d) optionally removing the proteins that have elicited antibodies in step (a) from the initial mixture to form a mixture of non-responding proteins; and (e) optionally repeating the previous steps (a) through (d) one or more times, each repetition utilizing the mixture of non-responding proteins from the immediate prior repetition in place of the initial mixture of proteins to identify one or more additional proteins present the tissue or fluid to be evaluated. Preferably, steps (a) through (d) are repeated one or more times, each repetition utilizing the mixture of non-responding proteins from the prior repetition in place of the initial mixture of proteins to identify one or more additional proteins present the tissue or fluid to be evaluated.

In some embodiments of the present method, the host organism in each repetition is from the same species. In other embodiments, the host organism from each successive repetition is from a different species than the host organism used in any prior repetition. In yet other embodiments, the host organism from each successive repetition is from a different species than the host organism used in at least one prior repetition.

Preferably, the contacting step is performed by immunoblotting an array of known proteins (e.g., a protein chip) with the antiserum of step (a). The removing step preferably is accomplished by antiserum affinity chromatography.

It is preferred that the tissue or fluid to be evaluated is from a human.

In another aspect, the present invention provides a method of preparing antisera for identifying proteins in a proteome comprising the steps of: (a) immunizing a host organism with a sample containing an initial mixture of proteins from a tissue or fluid to be evaluated to elicit an antibody response to one or more proteins in the initial mixture; (b) isolating a first antiserum from the host organism after immunization; (c) removing proteins that elicited antibody formation in step (a) from the initial mixture of proteins to form a first mixture of non-responding proteins; (d) repeating the previous steps (a) and (b) utilizing the first mixture of non-responding proteins from step (c) in place of the initial mixture of proteins to isolate a second antiserum; and (e) optionally repeating steps (a) through (d) one or more times to isolate one or more additional antiserum, each successive repetition utilizing in step (d) a mixture of non-responding proteins from the immediately prior repetition in place of the first mixture of non-responding proteins.

The antisera prepared by the present methods are useful for identifying proteins in a proteome. For example, an antiserum produced by the method can be contacted with a known protein to determine whether the protein reacts with an antibody in the antiserum. An antibody reaction between the known protein and the antiserum indicates that the known protein was present in the proteome of the tissue or fluid which was evaluated.

In some preferred embodiments, each repetition utilizes the same host organism, while in other embodiments, a different host organism is utilized for each repetition. It is preferred that the tissue or fluid be from a human.

Results

Illustration of the principle of ABCSP using 18-proteins model system.

To illustrate the principle of the ABCSP approach, a model system was used with a mixture of 18 randomly chosen commercially available proteins (Table 1). To mimic the vast difference in protein concentration in biological samples, the proteins were mixed unequally so that the rarest component only comprised about 0.02% of the total proteins and the richest component comprised about 67.61% (Table 1). According to the immunization amount, these proteins were divided into three groups: (I) 7 proteins in the range of 0.033 to 0.067 mg, each comprising about 0.022-0.045%; (II) 9 proteins in the range of 0.33 mg to 3.33 mg, each comprising about 0.22-2.2%; and (III) 2 proteins in the range of 33 mg and above, comprising about 22% and 68%, respectively, of the total.

The mixture of these 18 proteins was injected to rabbits as described herein. The antisera were harvested from three rabbits and mixed to minimize the individual difference, and then the microchip containing these 18 proteins was immunoblotted.

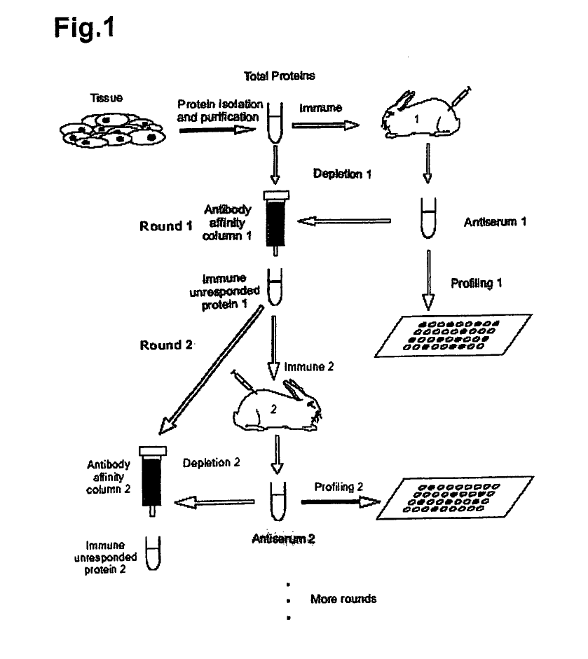

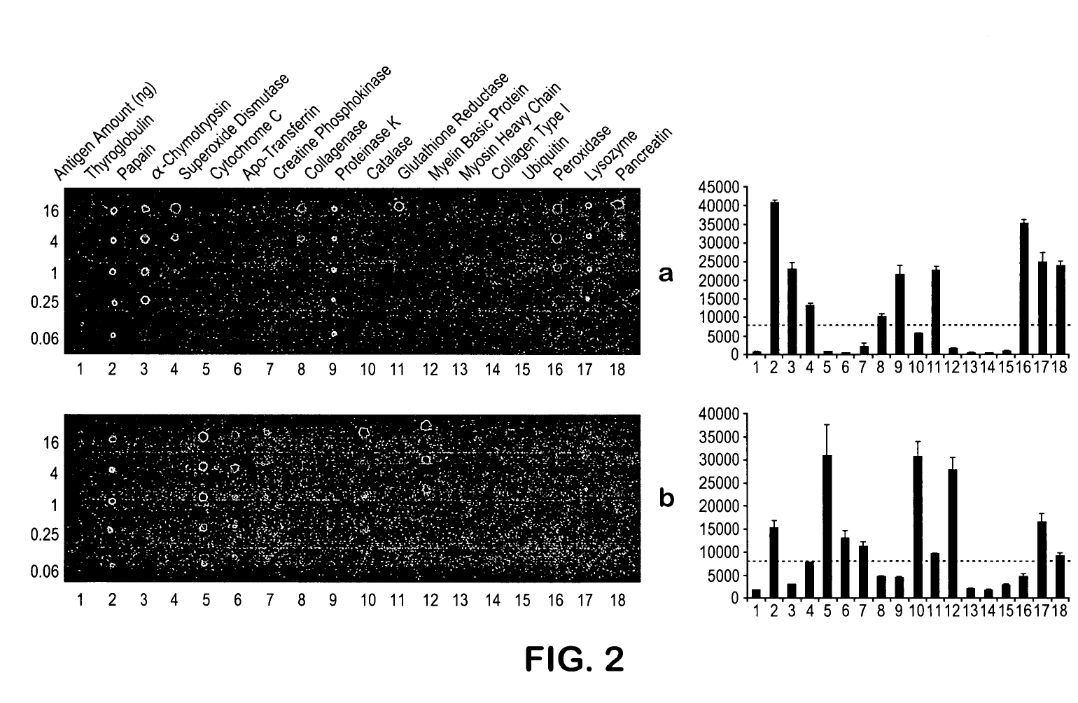

Among the 18 proteins, 9 showed positive signals (Table 1 and FIG. 1), i.e., 50% of protein induced antibodies. When the result was closely analyzed, 4 out of 7 (57%) proteins from group I had induced antibodies, indicating that even if the protein abundance was at 0.02%, antibodies were still elicited. In group II, 3 out of 9 proteins (33%) induced antibodies, and both proteins from group III yielded an antibody response.

Statistical analysis with Microsoft Excel showed that the correlation constant between the protein amount and antibody titer was below 0.28, indicating that no obvious correlation existed. These data are in concert with the immunological principle that whether a protein induces antibody in a host does not strictly depend on its abundance; the combination of concentration and antigenicity is important.

To further identify the protein components that did not elicit detectable antibodies (nonantibody-eliciting proteins), a second cycle of enrichment, immunization and chip screening was performed.

The original 18-protein mixture was subjected to repeated immunodepletion using an affinity column coupled with the antibody pool from the first cycle of immunization as described herein. The leftover nonantibody-eliciting proteins (flow-through) were concentrated and subjected to the next cycle of immunization in naive hosts.

Nine proteins showed positive signal when the microchip containing the 18 proteins was blotted with the second-round antibody pool (FIG. 1 and Table 1). Five previous nonantibody-eliciting proteins became positive in the second round. This result indicated that some proteins, which did not elicit antibodies when mixed with a large population of other proteins, could induce antibody when they were relatively enriched. Meanwhile, 4 previous antibody-eliciting proteins were positive again. One likely reason is that these proteins were not completely depleted. However, this does not affect the overall usefulness of this method but only increases the cycles of immunization required.

Together from two cycles of immunization, 14 out of 18 proteins in the model system were identified. Thus, this system is proven to be feasible, and it is reasonable to expect that, by repeating the cycles of immunization, immunoblotting protein chips, immunodepletion and re-immunizing, the majority of proteins in a proteome will be able to induce their corresponding antibodies and be identified eventually.

Antiserum induced by whole saliva recognized majority of saliva proteins detected in 2DE gel.

To determine whether this model can be applied to real proteomes, three rabbits were immunized each with 20 mg of total saliva proteins. The sera from three rabbits were pooled to minimize the individual difference. The total IgG was purified, and antibacteria components were removed by immunoabsorption with total bacterial proteins to eliminate the interference of antibacteria antibodies as described herein.

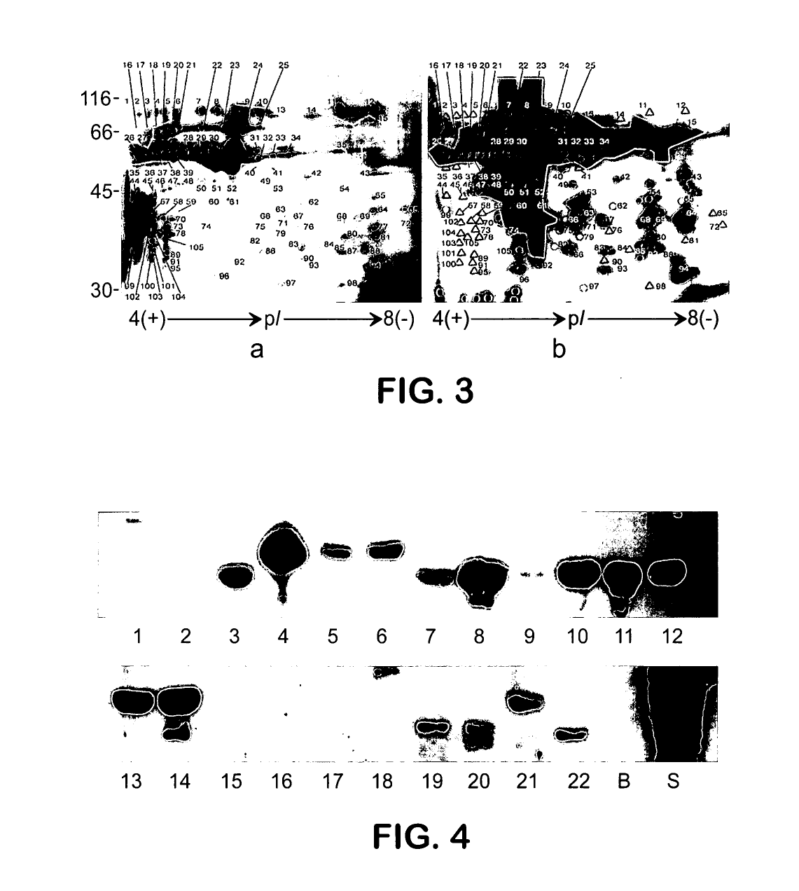

Two duplicated 2DE of saliva proteins were performed. One gel was subjected to silver staining; and the other was transferred to a PVDF membrane followed by Western blot with the purified total antisaliva antibodies (FIG. 3). The 2DE pattern of saliva is similar to most of the published data^{11,34}. Spots at the range of 50-70 kDa were crowded because various isoforms of amylase, albumin and immunoglobins are highly abundant in saliva”. In addition, to ensure the detection of as many low abundant proteins as possible by silver stain on the 2DE gel, much higher amounts of total saliva proteins (320 μg per gel) than most other reports (60 μg)³⁴ were loaded on the 2DE gels.

A total of 105 spots were visible on the silver stain gel (FIG. 3). Among them, 33 spots had distinct corresponding spots on Western blot membrane, 36 spots were located in the outlined area where the spots were not separated well on Western blot membrane, and another 36 spots did not have corresponding spots on Western blot. Therefore, approximately 34% (36/105) of silver staining visible proteins did not induce Western blot-detectable antibodies and needed further immunization to obtain corresponding antibody. In addition, 15 spots on Western blot did not have corresponding spots on silver staining signal. Apparently these 15 proteins were below the detection limit of silver stain, and thus their presence strongly suggests that this immunological approach could complement the 2DE-MS method by which only stained proteins can be detected.

However, the resolution of Western blot on a membrane is lower than that with silver staining on original gel, because when proteins are transferred to the membrane, they may diffuse. The visualization of protein spots with fluorescent method also resulted in diffusion of substrate and scattering of lights. Therefore, the protein spots in the outlined area (FIG. 3) did not appear well-separated on Western blot.

Anti-saliva aniserum contained antibodies recognizing most of the reported saliva proteins.

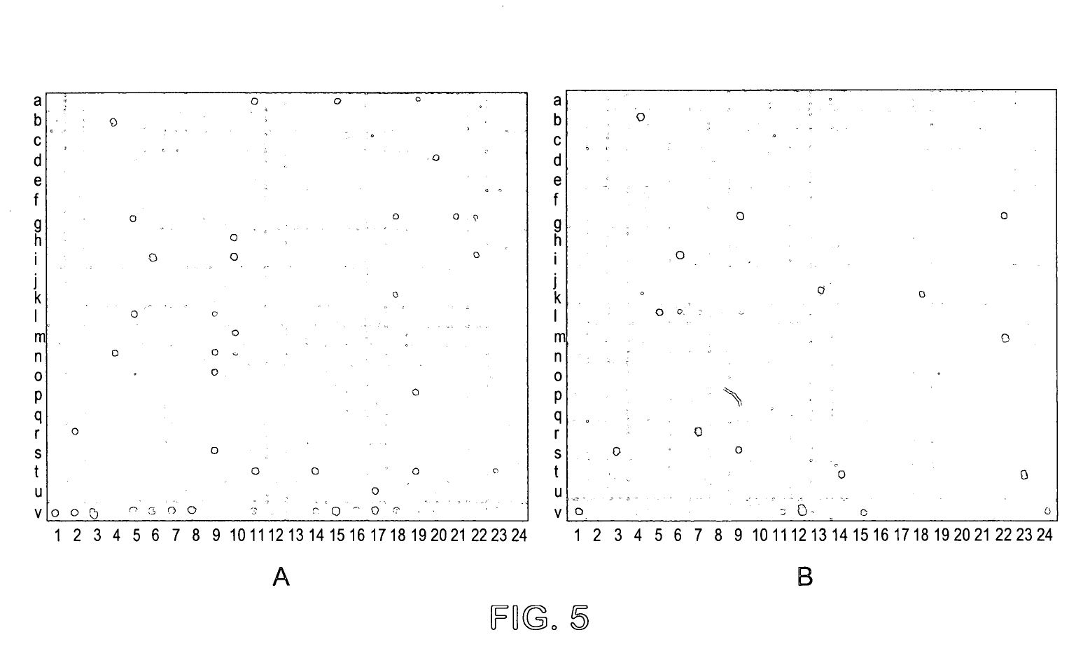

One of the best quality controls of the antiserum generated in this method is to examine whether the antiserum can recognize some previously reported saliva proteins. To do so, 22 known saliva proteins were expressed as GST-fusion proteins. The antisaliva antibody was tested against each of these proteins by Western blot (FIG. 4). The antisaliva antiserum recognized 18 out of 22 expressed saliva proteins (18/22=81.8%) on Western blot (FIG. 4, Table 2). This percentage was higher than that observed on 2DE Western blot, possibly because most previously identified proteins were silver-stained detectable spots, which means they have a higher abundance in total mixture.

New saliva proteins were identified with this new system.

Protein chips were prepared containing 504 randomly chosen GST fusion proteins from an EST (Express Sequence Tags) repertoire and the above described 22 known saliva proteins were used as a control. The protein chip was probed with our first round anti-saliva antisera. Among the 22 known saliva proteins, 20 (20/22=91%) showed the same results as Western blot (Table 2), indicating that protein chip screening was reliable. Among these 504 proteins, 29 showed positive with the antisaliva sera (Table 3) and these 29 proteins are all previously undiscovered saliva proteins. If a protein chip containing more standard proteins was available, it is likely that more undiscovered saliva proteins would be identified.

Since the first cycle of immunization yielded antibodies against an estimated 60% proteins, an affinity column was prepared with total IgG of anti-saliva sera to deplete these proteins. Total saliva proteins have been absorbed multiple times until no proteins bound to the column. The saliva proteins in flow through were then concentrated and subjected to a new cycle of immunization in naive hosts.

The antiserum resulted from second cycle of immunization was again used to screen the antigen chip containing the same 504 proteins. Fifteen proteins showed positive signals. Among them, 7 out of the 15 proteins were not observed in the first cycle of immunization (Table 3) and the other 8 proteins were previously identified by the first round serum, indicating most but not all of the antibody-eliciting proteins in the first round immunization were depleted. All together 36 new saliva proteins were identified.

To further confirm that these 36 proteins were indeed present in saliva, we searched and found antibodies against 15 proteins for confirmation with Western blot, which would better distinguish the real signal from background noise by the additional molecular weight information. In this experiment, total saliva proteins were subjected to SDS-PAGE followed by Western blot with each of the available antibodies. Twelve out of 15 of the identified new saliva proteins showed a positive signal (FIG. 4). The other 3 proteins were not detected by their corresponding antibodies suggesting either chip error, or low abundance in saliva below the detection limit for Western blot.

Discussion

The present methods provide a number of advantages for proteomic analysis including:

1) The antigen signals can be translated into and represented by antibody signals after sufficient cycles of immunization, immunoblot of protein chips, immunodepletion and re-immunization. In this process, the drastic difference in the protein amounts of a proteome can be roughly diminished or normalized either because the minor proteins have higher antigenicity or they were enriched and immunized to naive hosts.

2) A proteomic project can be carried out progressively. Daily progress will be recorded, and what has been screened can be set aside. The identified proteins will be accumulated in the database daily, and the focus will be on the unidentified proteins.

3) A single proteomic project can be carried out in parallel by several laboratories. As mentioned above, protein array preparation and screening can be performed in different laboratories, and the data can be combined to increase the time efficiency of the work and prevent redundant work.

Furthermore, the immunological method should also be a great complement to separation-based proteomics. The depleted proteins from each batch of serum can be subjected to 2DE (or LC)/MS, thus the complex proteome can be divided into several subgroups to be determined separately. The two approaches can be carried out separately or simultaneously.

It is relatively easy to prepare a large number of proteins according to this method because a very small amount of each protein is needed, and they do not need to be functionally active. They do not need to be full length and could be a collection of several fragments, which would make the expression work much easier. In addition, synthetic peptides can also be used to substitute the hard-to-express proteins. Currently, high throughput expression methods are developing quickly. For example, Zhu et al. expressed 5800 yeast GST-His6 fusion proteins in yeast to screen their interacting proteins³⁵; and LaBaer's group developed the high throughput self-assembling protein microarray technique³⁶. In addition, the construction of an expression library is cumulative. Once it is constructed, proteome screening is significantly faster and cheaper and it can be applied to any human proteomic project with the present methods.

The sensitivity and repeatability of detecting antibody by antigen chips should also be considered in this method. With the advent of the first commercial protein chip, the PROTOARRAY® protein chip from Invitrogen, large scale screening is possible Under proper conditions, an antibody and antigen microarray can detected with as little as 1 ng/ml protein or antibody².

Materials and Methods.

Materials: Purified proteins (listed in Table 1), mouse anti-GST (G-1160) antibody, Standard 1″×33″ microscope glass slides, complete and incomplete Freund's adjuvant were purchased from Sigma Aldrich (St. Louis, Mo.). QD-streptavidin (655 nm) was purchased from Quantum Dot Corp (Hayward, Calif.). Coomassie blue plus kit was purchased from Pierce Inc (Rockford, Ill.). Other polyclonal antibodies, 504 purified GST-fusion proteins, human full length genes were provided by Proteintech Group, Inc (Chicago, Ill.). Immunization of rabbits was also performed by Proteintech Group. CNBr-activated Sepharose 4B and Protein A Sepharose were purchased from Amersham Pharmacia Biotech (Piscataway, N.J.). Centricon-YM3 centrifugal filter devices were purchased from Millipore (Mass., USA, Cat. No.4202). BIO-LYTE® 3/10, BIO-LYTE® 8/10, BIO-LYTE® 3/5 and Silver Stain Plus kit were bought from BioRad (Hercules, Calif.). HRP or FITC labeled goat anti-rabbit or anti-mouse IgG were purchased from Jackson Immunoresearch Laboratories (West Grove, Pa.). Unstimulated whole saliva was collected from 4 healthy volunteers, 2 males and 2 females between the ages 20 and 40 (mean age 31). After tooth brushing for 3 min by using a tooth brush without toothpaste, the whole saliva samples were collected by spitting into ice-cooled sterile tubes. The collected saliva was centrifuged at 14,000g for 25 min and the supernatant was stored at −20 ° C.

Immunization:

Six-week-old New Zealand white rabbits were used for immunization. Each group of antigens was immunized into three rabbits. Primary injection was carried out in complete Freund's adjuvant. The rabbits were boosted at 2-week intervals with the same immunogen mixed with incomplete Freund's adjuvant 4 weeks after the first injection. Antisera of animals immunized with same antigen were harvested after 4 boosts and mixed together to minimize individual deviation.

Antibody Purification:

The total IgG from antisera was purified by protein A column according to the vender's protocol. To remove any anti-bacterial IgG, total IgG was immunoabsorbed over bacterial protein column multiple times until the antibody pool no longer reacted with bacterial lysate on ELISA. The bacterial proteins were harvested from an overnight culture of E. Coli Bl21 (DE3) strain in 50 ml LB medium by centrifugation and ultrasonication, reconstituted in coupling buffer (0.2 M NaHCO3, 0.5 M NaCl, pH 8.3) and coupled to CNBr-activated Sepharose. The coupled Sepharose was used to prepare a bacterial protein column.

Fusion Protein Expression:

Partial or whole coding region of saliva genes were amplified with PCR and cloned into pGEX-4T-3 vector in the E. Coli strain BL21 (DE3) as host (Table 2). GST fusion proteins were expressed and purified according to supplier's standard procedure and the expression was confirmed by SDS-PAGE.

Protein Microarray and Blotting:

Standard 1″×3″ microscope glass slides were activated with glycidyloxipropyltrimethoxysilane (GOPTS) as previously described³⁸. Proteins were dissolved in PBS (PH 7.4) with 40% glycerol spotted on chip with 1 nl per spot,

The printing of protein microarrays on GOPTS-activated glass slides were performed with PixSys 5500 spotting robot (Cartesian Technology, Irvine, Calif.) mounted with Arraylt SMP3 micro spotting pin from TeleChem (Sunnyvale, Calif.).

Immediately before use, the protein microarray slides were blocked with 1% BSA/PBS for 1 h at room temperature. Then the slides were rinsed in PBST (PBS with 0.1% Tween 20), PBS, ddH₂O (doubly distilled water) in turn each for 5 min. The slides were dried by centrifugation at 3000 rpm for 2 min.

The protein chip was first incubated with 20 μl of diluted antibodies for 1 hour at room temperature. And then the slides were washed with PBST, PBS, ddH₂O in turn each for 5 min to remove all unbound molecules. After that, the slides were probed with the biotin-conjugated goat anti-rabbit IgG for 1 h at room temperature. For detection, the microarray was incubated with 2 nM streptavidin-Qdot 655 for 1 h at room temperature. After a thorough washing as mentioned above, the microarray was scanned on a PerkinElmer ScanArray 5000 Scanner with the laser 1 (633 nm) and filter 8, power at 80%, photomultiplier at 80%, and a scan resolution of 5 mm.

Protein Depletion and Re-immunization:

Total IgG was coupled to CNBr-activated Sepharose 4B to prepare an affinity column. Protein mixture was passed through the column to remove those antigens which had induced antibodies. The flow through were then subjected to the next round depletion after the column was eluted with 0.1 M glycine (pH 3.0). Such depletion was repeated multiple times until no more protein in elution can be detected by Pierce Coomassie blue plus kit. The final flow through was used to immunize another 3 rabbits by the procedure mentioned above.

Two-dimensional Electrophoresis:

A saliva sample was four times concentrated with a Centricon-YM3 column. 40 μl concentrated saliva (about 320 μg) was used to prepare the loading sample with 42% Urea, 0.5% BIO-LYTE®, 1% B-mecaptoethanol and applied to the IEF electrophoresis. The IEF gel contained 4% acrylamide, 0.2% bis-acrylamide, 55% urea, 5% BIO-LYTE® 3/10 and 2% NP-40. The IEF electrophoresis was run at 4 watts for a total of 6000 V-h, with the voltage ramped to about 1200 V. After IEF, the first dimension gel stripes were immediately separated with 14% SDS-PAGE. The resulted two-dimensional gel was either stained with Silver Stain Plus kit or subjected to Western blotting with the mixed antiserum from the saliva-immunized rabbits.

Abbreviations:

ABCSP: antibody based cumulative subtraction proteomics

2DE: two-dimensional gel electrophoresis

LC: liquid chromatograph

MS: mass spectrometry

In the Following Tables:

“(F)” indicates the full length gene was expressed.

“+” or “−” indicates the positive or negative result of Western blot or protein chip screening with the anti-saliva serum

“/” indicates that the antibodies were not available for Western blot confirmation.

“−” or “+” indicates that the Western blot confirmation produced a negative or positive result respectively.

TABLE 1
Summary of the model system with 18 pure proteins

| | | | | | Second round |
|---|---|---|---|---|---|
| | | First round immunization | | | Immunization |
| | Order | Ag Amt. per | Abundance | Mean Chip | Mean Chip |
| Antigen Name | on chip | Rabbit (mg) | (%) | Signal | Signal |

Group I

| | | | | | | | |
|---|---|---|---|---|---|---|---|
| Superoxide Dismutase | 4 | 0.033 | 0.02 | 13168 | (+) | 7745 | (−) |
| Glutathione Reductase | 11 | 0.033 | 0.02 | 22809 | (+) | 9667 | (+) |
| Myelin Basic Protein | 12 | 0.033 | 0.02 | 1607 | (−) | 27942 | (+) |
| Myosin Heavy Chain | 13 | 0.033 | 0.02 | 646 | (−) | 2135 | (−) |
| Collagen Type 1 | 14 | 0.033 | 0.02 | 540 | (−) | 1783 | (−) |
| peroxidase | 16 | 0.033 | 0.02 | 35401 | (+) | 4899 | (−) |
| Proteinase K | 9 | 0.067 | 0.05 | 21514 | (+) | 4492 | (−) |

Group II

| | | | | | | | |
|---|---|---|---|---|---|---|---|
| Apo-Transferrin | 6 | 0.333 | 0.23 | 576 | (−) | 13125 | (+) |
| Creatine phosphokinase | 7 | 0.333 | 0.23 | 2395 | (−) | 11290 | (+) |
| Ubiquitin | 15 | 1 | 0.68 | 1114 | (−) | 3011 | (−) |
| Collagenase | 8 | 1.667 | 1.13 | 10179 | (+) | 4638 | (−) |
| α-Chymotrypsin | 3 | 1.667 | 1.13 | 23081 | (+) | 3066 | (−) |
| Thyroglobulin | 1 | 2 | 1.35 | 822 | (−) | 1971 | (−) |
| Cytochrome C | 5 | 2 | 1.35 | 977 | (−) | 30976 | (+) |
| Catalase | 10 | 2 | 1.35 | 5739 | (−) | 30766 | (+) |
| Papain | 2 | 3.333 | 2.25 | 41003 | (+) | 15336 | (+) |

Group III

| | | | | | | | |
|---|---|---|---|---|---|---|---|
| Lysozyme | 17 | 33.333 | 22.54 | 24965 | (+) | 16626 | (+) |
| Pancreatin | 18 | 100 | 67.61 | 24008 | (+) | 9369 | (+) |

TABLE 2
The reported saliva proteins used to evaluate anti-saliva protein serum

| | Protein | Swiss- | Expressed | Western | Chip | Position | |
|---|---|---|---|---|---|---|---|
| NO. | Name | Prot No. | region | blot | Signal | on chip | Ref. |
| 1 | AMY2B | P19961 | N-190aa | + | + | (V, 2) | 39 |
| 2 | B2M | Q6IAT8 | 119aa(F) | − | + | (V, 14) | 9, 40 |
| 3 | S100A8 | P05109 | 93aa(F) | + | + | (V, 3) | 9 |
| 4 | S100A9 | P06702 | 114aa(F) | + | + | (V, 15) | 9 |
| 5 | ENO1 | P06733 | N-212aa | + | − | (V, 4) | 40 |
| 6 | ACATE2 | Q96EA2 | 212aa(F) | + | + | (V, 16) | 9 |
| 7 | FABP1 | P07148 | 127aa(F) | + | + | (V, 5) | 9 |
| 8 | FABP3 | P05413 | 133aa(F) | + | + | (V, 17) | 9 |
| 9 | FABP4 | P15090 | 112aa(F) | + | + | (V, 6) | 9 |
| 10 | FABP5 | Q01469 | 135aa(F) | + | + | (V, 18) | 9 |
| 11 | FABP6 | P51161 | 128aa(F) | + | + | (V, 7) | 9 |
| 12 | FABP7 | O15540 | 132aa(F) | + | + | (V, 19) | 9 |
| 13 | GSTA1 | P08263 | 222aa(F) | + | + | (V, 8) | 9, 40 |
| 14 | GSTA4 | O15217 | 222aa(F) | + | + | (V, 20) | 9, 40 |
| 15 | KLK1 | P06870 | 262aa(F) | − | − | (V, 9) | 31 |
| 16 | KLK2 | P20151 | 261aa(F) | − | − | (V, 21) | 31 |
| 17 | KLK8 | O60259 | 260aa(F) | − | − | (V, 10) | 31 |
| 18 | LIPA | P38571 | N-339aa | + | + | (V, 22) | 33 |
| 19 | LYZ | P61626 | 148aa(F) | + | + | (V, 11) | 34, 41 |
| 20 | PIP | P12273 | 146aa(F) | + | + | (V, 23) | 9 |
| 21 | PRL | P01236 | 227aa(F) | + | + | (V, 12) | 9 |
| 22 | Hbb | Q14477 | 147aa(F) | + | + | (V, 24) | 10 |
| 23 | BL21 | | | − | − | (V, 13) | |
| 24 | Saliva | | | + | + | (V, 1) | |

TABLE 3
The newly identified saliva proteins by antigen microarray

| | | | |
|---|---|---|---|
| | | Chip | West Blot |
| Protein Name | SWISS-PROT | position | Confirmation |

1^{st} round

| | | | |
|---|---|---|---|
| KPNB1 | Q14974 | a, 11 | / |
| CDC37 | Q16543 | a, 15 | + |
| SSR2 | P43308 | a, 19 | / |
| DDX50 | Q9BQ39 | b, 4 | / |
| ENO2 | P09104 | d, 20 | + |
| ENIGMA | Q9NR12 | g, 5 | / |
| HDAC3 | O15379 | g, 18 | / |
| MMP9 | P14780 | g, 21 | + |
| KRT15 | P19012 | g, 22 | + |
| MKNK1 | Q9BUB5 | h, 10 | + |
| ICAM2 | P13598 | i, 6 | / |
| APEX1 | P27695 | i, 10 | + |
| S100A11 | P31949 | i, 22 | / |
| POU2F1 | P14859 | k, 18 | − |
| NFKBIA | P25963 | l, 5 | / |
| POLD2 | P49005 | l, 9 | / |
| ZYX | Q15942 | m, 10 | + |
| RNH | Q9BQ80 | n, 4 | / |
| lasp1 | Q14847 | n, 9 | / |
| CSTF1 | Q05048 | n, 10 | / |
| CAPG | P40121 | o, 9 | + |
| UBE2I | P63279 | p, 19 | + |
| S100A6 | P06703 | r, 2 | / |
| HPRP3P | O43395 | s, 9 | / |
| DHX38 | Q92620 | t, 11 | + |
| MADH1 | Q15797 | t, 14 | / |
| EXT1 | Q16394 | t, 19 | / |
| PVRL2 | Q92692 | t, 23 | / |
| MATK | P42679 | u, 17 | / |

2^{nd} round

| | | | |
|---|---|---|---|
| PPP1CB | P62140 | g, 9 | / |
| UBB | Q96H31 | k, 13 | + |
| H3F3B | P84243 | l, 6 | − |
| PLAT | Q9BU99 | l, 7 | / |
| GAPD | P04406 | m, 22 | + |
| CX3CL1 | P78423 | r, 7 | / |
| DYNACTIN 4 | Q9BTE1 | s, 3 | − |

Reference List

1. Pandey, A. & Mann, M. Proteomics to study genes and genomes. Nature 405: 837-846 (2000).

2. Haab, B. B., Dunham, M. J., & Brown, P. O. Protein microarrays for highly parallel detection and quantitation of specific proteins and antibodies in complex solutions. Genome Biol. 2: RESEARCH0004 (2001).

3. Gygi, S. P., Rochon, Y., Franza, B. R., & Aebersold, R. Correlation between protein and mRNA abundance in yeast. Mol. Cell Biol. 19: 1720-1730 (1999).

4. Anderson, L. & Seilhamer, J. A comparison of selected mRNA and protein abundances in human liver. Electrophoresis 18: 533-537 (1997).

5. Olsen, J. V. & Mann, M. Improved peptide identification in proteomics by two consecutive stages of mass spectrometric fragmentation. Proc. Natl. Acad. Sci. U.S.A. 101: 13417-13422 (2004).

6. Aebersold, R. & Mann, M. Mass spectrometry-based proteomics. Nature 422: 198-207 (2003).

7. Skop, A. R., Liu, H., Yates, J., Meyer, B. J., & Heald, R. Dissection of the mammalian midbody proteome reveals conserved cytokinesis mechanisms. Science 305: 61-66 (2004).

8. Cronshaw, J. M., Krutchinsky, A. N., Zhang, W., Chait, B. T., & Matunis, M. J. Proteomic analysis of the mammalian nuclear pore complex. J Cell Biol. 158: 915-927 (2002).

9. Ghafouri, B., Tagesson, C., & Lindahl, M. Mapping of proteins in human saliva using two-dimensional gel electrophoresis and peptide mass fingerprinting. Proteomics; 3(6):1003-1015 (2003)

10. Huang, C. M. Comparative proteomic analysis of human whole saliva. Arch. Oral Biol; 49(12):951-962 (2004).

11. Vitorino, R. et al. Identification of human whole saliva protein components using proteomics. Proteomics 4: 1109-1115 (2004).

12. Petricoin III, E. F. et al. Use of proteomic patterns in serum to identify ovarian cancer. The Lancet 359: 572-577 (2002).

13. Coombes, K. R., Morris, J. S., Hu, J., Edmonson, S. R., & Baggerly, K. A. Serum proteomics profiling—a young technology begins to mature. Nat. Biotechnol; 23(3):291-292 (2005).

14. Oh, P. et al. Subtractive proteomic mapping of the endothelial surface in lung and solid tumours for tissue-specific therapy. Nature; 10:629-635 (2004).

15. Khidekel, N., Ficarro, S. B., Peters, E. C., & Hsieh-Wilson, L. C. Exploring the O-GlcNAc proteome: Direct identification of O-GlcNAc-modified proteins from the brain. Proc. Natl. Acad. Sci. U.S.A.; 101(36):13132-13137 (2004).

16. Pisitkun, T., Shen, R. F., & Knepper, M. A. Identification and proteomic profiling of exosomes in human urine. Proc. Natl. Acad. Sci. U S. A. 101(36):13368.-13373 (2004).

17. Kabuyama, Y., Resing, K. A., & Ahn, N. G. Applying proteomics to signaling networks. Curr. Opin. Genet. Dev.; 14(5):492-498 (2004).

18. Santoni, V., Molloy, M., & Rabilloud, T. Membrane proteins and proteomics: un amour impossible? Electrophoresis; 21(6):1054-1070 (2000).

19. Pavlik, P. et al. Predicting antigenic peptides suitable for the selection of phage antibodies. Hum. Antibodies; 12(4):99-112 (2003).

20. Santoni, V., Molloy, M., & Rabilloud, T. Membrane proteins and proteomics: un amour impossible? Electrophoresis 21: 1054-1070 (2000).

21. Rabilloud, T. Two-dimensional gel electrophoresis in proteomics: old, old fashioned, but it still climbs up the mountains. Proteomics. 2: 3-10 (2002).

22. Garbis, S., Lubec, G., & Fountoulakis, M. Limitations of current proteomics technologies. J Chromatogr. A 1077: 1-18 (2005).

23. Huang, H. L. et al. Enrichment of low-abundant serum proteins by albumin/immunoglobulin G immunoaffinity depletion under partly denaturing conditions. Electrophoresis 26: 2843-2849 (2005).

24. Hinerfeld, D., Innamorati, D., Pirro, J., & Tam, S. W. Serum/Plasma depletion with chicken immunoglobulin Y antibodies for proteomic analysis from multiple Mammalian species. J Biomol. Tech. 15: 184-190 (2004).

25. Tishler, M., Yaron, I., Shirazi, I., & Yaron, M. Saliva: an additional diagnostic tool in Sjogren's syndrome. Semin. Arthritis Rheum. 27: 173-179 (1997).

26. Beeley, J. A. & Khoo, K. S. Salivary proteins in rheumatoid arthritis and Sjogren's syndrome: one-dimensional and two-dimensional electrophoretic studies. Electrophoresis 20: 1652-1660 (1999).

27. Dutta, S. K., Dukehart, M., Narang, A., & Latham, P. S. Functional and structural changes in parotid glands of alcoholic cirrhotic patients. Gastroenterology 96: 510-518 (1989).

28. Shori, D. K. et al. Altered sialyl- and fucosyl-linkage on mucins in cystic fibrosis patients promotes formation of the sialyl-Lewis X determinant on salivary MUC-5B and MUC-7. Pflugers Arch. 443 Suppl 1, S55-S61(2001).

29. Hagewald, S. J., Fishel, D. L., Christan, C. E., Bernimoulin, J. P., & Kage, A. Salivary IgA in response to periodontal treatment. Eur. J Oral Sci. 111: 203-208 (2003).

30. Filaire, E., Duche, P., & Lac, G. Effects of training for two ball games on the saliva response of adrenocortical hormones to exercise in elite sportswomen. Eur. J Appl. Physiol Occup. Physiol 77: 452-456 (1998).

31. Streckfus, C. F. & Bigler, L. R. Saliva as a diagnostic fluid. Oral Dis.; 8(2):69-76 (2002).

32. Henskens, Y. M. et al. Cystatins S and C in human whole saliva and in glandular salivas in periodontal health and disease. J Dent. Res. 73: 1606-1614 (1994).

33. Kaufman, E. & Lamster, I. B. Analysis of saliva for periodontal diagnosis—a review. J. Clin. Periodontol.; 27(7):453-465 (2000).

34. Yao, Y., Berg, E. A., Costello, C. E., Troxler, R. F., & Oppenheim, F. G. Identification of protein components in human acquired enamel pellicle and whole saliva using novel proteomics approaches. J. Biol. Chem.; 278(7):5300-5308 (2003).

35. Zhu, H. et al. Global analysis of protein activities using proteome chips. Science 293: 2101-2105 (2001).

36. Ramachandran,N. et al. Self-assembling protein microarrays. Science; 305:86-90 (2004)

37. Sheridan, C. Protein chip companies turn to biomarkers. Nat. Biotechnol.; 23(1):3-4 (2005).

38. Liang, R. Q., Tan, C. Y., & Ruan, K. C. Colorimetric detection of protein microarrays based on nanogold probe coupled with silver enhancement. Journal of Immunological Methods 285: 157-163 (2004).

39. Patton, J. R. & Pigman, W. Amylase in electrophoretic and ultracentrifugal patterns of human parotid saliva. Science 125: 1292-1293 (1957).

40. Warner, T. D. & Mitchell, J. A. Nonsteroidal antiinflammatory drugs inhibiting prostanoid efflux: as easy as ABC? Proc. Natl. Acad. Sci. U.S.A.; 100(16):9108-9110 (2003).

41. Strober, W., Fuss, I., & Kitani, A. Regulation of experimental mucosal inflammation. Acta Odontol. Scand.; 59(4):244-247 (2001).

Numerous variations and modifications of the embodiments described above can be effected without departing from the spirit and scope of the novel features of the invention. It is to be understood that no limitations with respect to the specific embodiments illustrated herein are intendend or should be inferred. It is, of course, intended to cover by the appended claims all such modifications as fall within the scope of the claims.

What is claimed is:

1. A method of identifying proteins in a proteome comprising the steps of:
(a) immunizing a host organism with a sample containing an initial mixture of proteins from a tissue or fluid to be evaluated to elicit an antibody response to one or more proteins in the initial mixture;
(b) isolating an antiserum from the host organism after immunization;
(c) contacting the antiserum with an array of known proteins to form one or more antibody-protein conjugates, thereby identifying the one or more proteins in the initial mixture that elicited an antibody response in the host organism;
(d) optionally removing the proteins that elicited antibody formation in step (a) from the initial mixture to form a mixture of non-responding proteins; and
(e) optionally repeating the previous steps (a) through (d) one or more times, each successive repetition utilizing in step (a) the mixture of non-responding proteins from the immediate prior repetition in place of the initial mixture of proteins to identify one or more additional proteins present in the tissue or fluid to be evaluated.

2. The method of claim 1 wherein steps (a) through (d) are repeated one or more times, each repetition utilizing the mixture of non-responding proteins from the preceding repetition in place of the initial mixture of proteins to identify one or more additional proteins present the tissue or fluid to be evaluated.

3. The method of claim 2 wherein the host organism used in each repetition is from the same species.

4. The method of claim 2 wherein the host organism from each successive repetition is from a different species than the host organism used in any prior repetition.

5. The method of claim 2 wherein the host organism from each successive repetition is from a different species than the host organism used in at least one prior repetition.

6. The method of claim 1 wherein the contacting is performed by immunoblotting the array of known proteins with the antiserum.

7. The method of claim 6 wherein the array of known proteins is a protein chip.

8. The method of a claim 1 wherein the removing is accomplished by antiserum affinity chromatography.

9. The method of claim 1 wherein the tissue or fluid to be evaluated is from a human.

10. An isolated protein identified by the method of claim 1.

11. An isolated antibody to a protein of claim 10.

12. A method of preparing antisera for identifying proteins in a proteome comprising the steps of:
(a) immunizing a host organism with a sample containing an initial mixture of proteins from a tissue or fluid to be evaluated to elicit an antibody response to one or more proteins in the initial mixture;
(b) isolating a first antiserum from the host organism after immunization;
(c) removing proteins that elicited antibody formation in step (a) from the initial mixture of proteins to form a first mixture of non-responding proteins;
(d) repeating the previous steps (a) and (b) utilizing the first mixture of non-responding proteins from step (c) in place of the initial mixture of proteins to isolate a second antiserum; and
(e) optionally repeating steps (a) through (d) one or more times to isolate one or more additional antiserum, each successive repetition utilizing in step (d) a mixture of non-responding proteins from the immediately prior repetition in place of the first mixture of non-responding proteins.

13. The method of claim 13 wherein a different host organism is utilized in each repetition.

14. The method of claim 12 wherein the removing in step (c) is accomplished by antiserum affinity chromatography.

15. The method of claim 12 wherein the tissue or fluid is from a human.

16. The method of claim 16 wherein the tissue or fluid is saliva.

17. An antiserum prepared by the method of claim 17.

18. An antiserum prepared by the method of claim 12.
